(19) United States
(12) Patent Application Publication
(10) Pub. No.: US 2026/0053441 A1
(43) Pub. Date: Feb. 26, 2026

(54) SMART DEVICES AND ASSOCIATED METHODS FOR ORTHOPEDIC PROCEDURES

(71) Applicant: Arthrex, Inc., Naples, FL (US)

(72) Inventors: Jonathan Knopf, Naples, FL (US); Robert K. Fergan, Naples, FL (US)

(73) Assignee: Arthrex, Inc., Naples, FL (US)

(21) Appl. No.: 19/102,550

(22) PCT Filed: Feb. 22, 2024
(86) PCT No.: PCT/IB2023/058203

§ 371 Date: Feb. 10, 2025

Related U.S. Application Data
(60) Provisional application No. 63/398,049, filed on Aug. 15, 2022.

(51) Int. Cl. A61B 5/00; A61B 17/04; A61B 90/00

(52) U.S. Cl. CPC A61B 5/6812; A61B 5/7475; A61B 17/0401; A61B 90/06; A61B 2090/061; A61B 2090/067

(57) ABSTRACT

An apparatus for orthopedic surgery includes at least one surgical accessory connected to an anatomical structure. The surgical accessory includes a first sensor and a second sensor connected to spatially separated portions of the anatomical structure. A controller is in communication with the first sensor and the second sensor. The controller is configured to detect a first sensor and a second position of the second sensor in a common coordinate system. The controller identifies a spatial relationship between the first position and the second position and detects a change in the spatial relationship. In response to the change in the relationship, the controller outputs an indication reporting a movement of a portion of the surgical accessory relative to the at least one anatomical structure.

### BACKGROUND

The present disclosure generally relates to devices and sensors for use in orthopedic procedures and, more particularly, relates to medical devices and implements configured to intraoperatively or postoperatively measure or detect characteristics of the medical devices. Tracking the procedure and execution of an orthopedic procedure may allow health care providers to improve medical procedures by educating physicians and providing comparative data related to patient outcomes. Additionally, periodic or ongoing monitoring of surgical accessories and devices implanted in a medical procedure may inform care providers of various milestones or complications to assist in rehabilitation. The disclosure provides for devices and methods that may introduce sensory devices in surgical implants and accessories related to orthopedic procedures to improve patient care.

### SUMMARY

An apparatus for orthopedic surgery comprises at least one surgical accessory that connects to at least one anatomical structure. The at least one surgical accessory includes a first sensor and a second sensor connected to spatially separated portions of the at least one anatomical structure. A controller is in communication with the at least one surgical accessory and configured to detect a first position of the first sensor in a common coordinate system and a second position of the second sensor in the common coordinate system. The controller then identifies a spatial relationship between the first position and the second position and detects a change in the spatial relationship. Once detected, the controller outputs an indication reporting the change in the relationship as a diagnostic indication of a movement of a portion of the surgical accessory moved relative to the at least one anatomical structure.

A method for detecting a condition of an orthopedic assembly comprises attaching the orthopedic assembly to a first portion of an anatomical structure with a first accessory including a first sensor and attaching the orthopedic assembly to a second portion, spatially separated from the first portion of the anatomical structure, with a second accessory including a second sensor. The method then identifies a first position of the first sensor in a common coordinate system and a second position of the second sensor in the common coordinate system. After determining a spatial relationship between the first position and the second position, the method is able to detect a change in the spatial relationship and a change in a fixation state of a surgical assembly relative to the anatomical structure in response to the change in the spatial relationship.

A tissue repair apparatus is disclosure that includes a fixation device comprising an anchor configured to engage a bone tunnel of a bone. The anchor comprises a body with an interior passage. A suture is in connection with the anchor and extends through the interior passage. The suture forms an enclosed loop that captures tissue and secures the tissue relative to the bone. At least one connection sensor is attached to or otherwise formed as a portion of the suture. The wherein the at least one sensor is configured to detect a force applied to the suture and wirelessly communicate the force as sensor data. A method for installing a tissue repair apparatus is also disclosed.

These and other features, objects and advantages of the present disclosure will become apparent upon reading the following description thereof together with reference to the accompanying drawings.

### BRIEF DESCRIPTION OF THE DRAWINGS

FIG. 1 illustrates devices of a surgical system including sensors situated relative to tissue;

FIG. 2 illustrates an implant system including sensors situated relative to components of the implant system;

FIG. 3 illustrates a sensor coupled to a component of the implant system of FIG. 2 at a frangible connection;

FIG. 4 illustrates sensors positioned relative to suture and an anchor;

FIG. 5A illustrates a first exemplary sectional view taken along line V-V of the suture of FIG. 4;

FIG. 5B illustrates a second exemplary sectional view taken along line V-V of the suture of FIG. 4;

FIG. 5C illustrates a third exemplary sectional view taken along line V-V of the suture of FIG. 4;

FIG. 5D illustrates a fourth exemplary sectional view taken along line V-V of the suture of FIG. 4;

FIG. 5E illustrates a fifth exemplary sectional view taken along line V-V of the suture of FIG. 4;

FIG. 5F illustrates a sixth exemplary view of the suture of FIG. 4 demonstrating a connecting feature extending along longitudinal section of the suture;

FIG. 6A illustrates smart devices for orthopedic procedures and associated methods;

FIG. 6B illustrates a system comprising a controller configured to communicate with one or more sensors of smart devices for orthopedic procedures and associated methods;

FIG. 7 illustrates sensors positioned relative to suture, anchors and an implant system;

FIG. 8 illustrates sensors positioned relative to suture, anchors and an implant system;

FIG. 9 illustrates an instrument incorporating sensors;

FIG. 10A illustrates a procedural example of an anchor and suture including one or more sensors;

FIG. 10B illustrates a procedural example of an anchor and suture including one or more sensors;

FIG. 11A illustrates a procedural example of an anchor and suture including one or more sensors;

FIG. 11B illustrates a procedural example of an anchor and suture including one or more sensors; and

FIG. 12 illustrates a surgical mesh comprising one or more sensors.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

In general, the disclosure provides for surgical accessories and devices that incorporate one or more sensor devices configured to report patient data that may be implemented intraoperatively and/or postoperatively. As provided in various detailed examples, the sensor devices (e.g., smart devices) may be implemented during surgical procedures to provide guidance and/or feedback to a physician regarding the placement (e.g., position and orientation) of an implant or tool to accurately place the implant or implement the tool. Intraoperative applications of the disclosed surgical accessories may also include a verification of a placement of an implant or accessory relative to a patient anatomy and monitoring characteristics detected at the surgical site. Following the completion of a procedure, the disclosed devices and systems may provide for monitoring of the implant or accessory and the corresponding anatomy (e.g., bones, tendons, etc.) during the recovery of a patient and throughout the life of the implant or accessory. In various examples, the disclosed surgical accessories or devices may report position information from one or more implanted accessories as well as environmental information indicating the local conditions (e.g., temperature, pressure, forces, blood flow, etc.) around the accessory and the localized environment. This information may be utilized to determine changes in the performance (e.g., fixation, rigidity, support tension, etc.) of the surgical accessory or implant as well as the local patient anatomy to which the accessory is attached. This information may assist health care providers in diagnosing patient conditions as well as improving the efficacy of procedures.

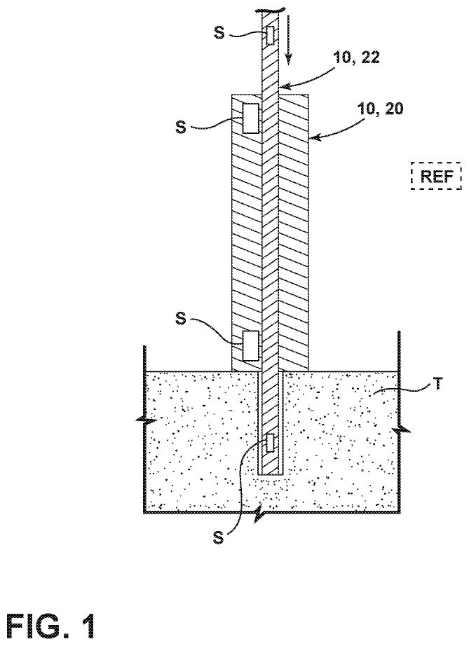

Referring generally to FIGS. 1-12, the disclosure provides for various surgical devices or accessories 10 that may incorporate monitoring circuits comprising one or more sensors S and transmission circuits (e.g., transceivers, emitters, etc.) configured to communicate sensor data to a monitoring system. Depending on the application, the monitoring system may correspond to a device controller for one or more surgical control consoles that may be configured to operate corresponding surgical tools (e.g., shavers, drills, electrosurgical probes, pumps, etc.). In some cases, the monitoring system may correspond to a diagnostic computer (e.g., tablet, kiosk, base station, etc.) that may receive the sensor data from the accessories 10 for local diagnostics or remote monitoring. In general, the surgical accessories 10 or devices may be incorporated into instruments and/or implants to detect or monitor various measurements, positions, orientations, and/or characteristics of the implant or instrument reporting the status of the surgical site before, during, or after a surgical procedure.

In various examples, the sensors S may be utilized to provide position or motion tracking of patient anatomy or to identify positions of the accessories 10 (e.g., anchors, sutures, implants, etc.). Position tracking may be provided by communicating with one or more of the sensors S via a receiver of the monitoring system to establish a location grid that may define a global coordinate system. To distinguish among multiple sensors S, each sensor S may be assigned a unique frequency or identification that may be tracked by the monitoring system to allow complex, multi-location tracking among sensors S. For example, in cases where the sensors S comprise accelerometers or inertial measurement units, each sensor S may report the relative position and orientation of the corresponding accessory 10 or portions of the surgical device/implant in six degrees of freedom to define the relative motion of multiple bodies. Additionally, an array of sensors S may establish and provide monitoring information for a rigid body or body designed for complex bending or movement. In this way, the sensors S may be utilized to determine if and the extent to which the accessories or corresponding anatomical structures shift or reorient following the implantation. Such information may be indicative of potential complications or recovery milestones that may inform care providers of conditions requiring correction or representative of healing to improve patient care.

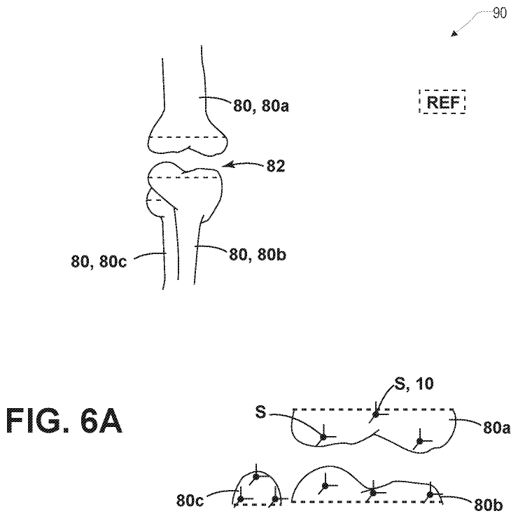

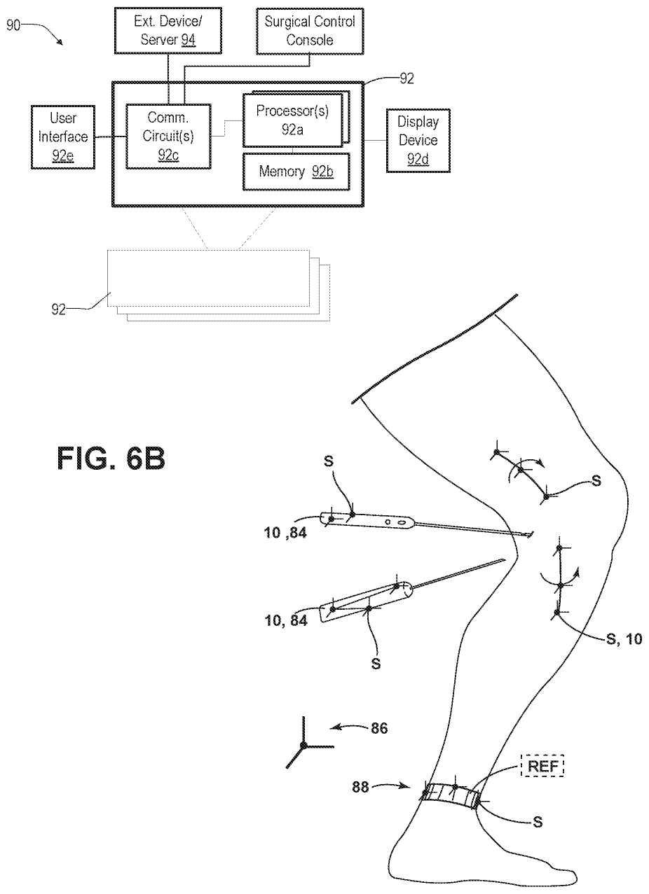

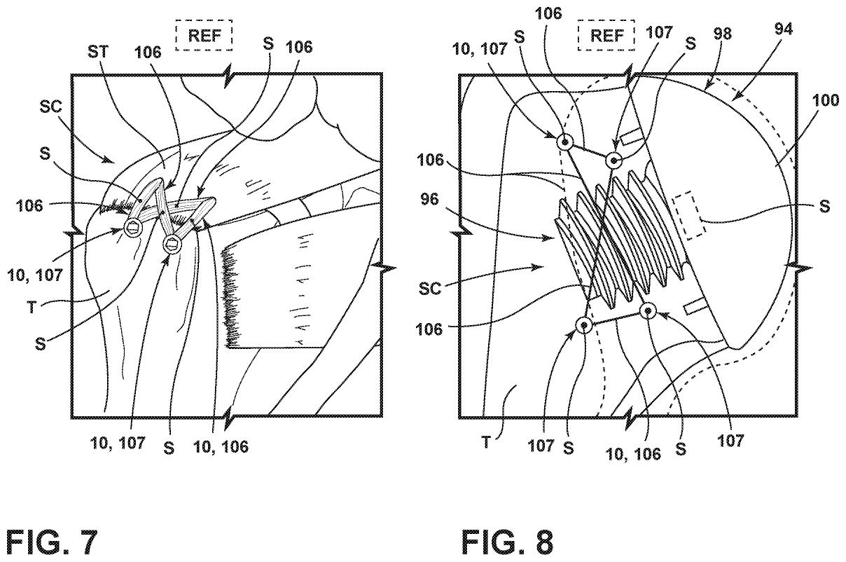

As later described in reference to FIG. 6, measurements of adjacent sensors can be compared to determine a range of motion (ROM). Additionally, FIGS. 7 and 8 provide detailed examples of how the relative motion or changes in the position among two or more of the sensors can be detected. Sensor information associated with ROM or positions of the sensors S may be tracked and evaluated over time to predict when a patient may substantially achieve full mobility, verify fixation, or otherwise monitor the positions of the surgical accessories 10. For example, a monitoring system may detect changes (e.g., increases, decreases, etc.) in ROM over a period (e.g., days, weeks, months) of time and may be compared to a model to predict or estimate when the patient may substantially achieve full mobility. The model may be based on usage, pathology and/or one or more parameters specified in the preoperative plan (e.g., procedure, implant type, etc.). Accordingly, the data reported by the sensors may be utilized to monitor the status of a patient as well as the status of the corresponding surgical accessories 10 and characteristics of the associated anatomy.

In addition to position or motion sensors (e.g., accelerometers, inertial measurement units, etc.), the characteristics of the patient anatomy and/or the surgical device or accessory may be detected and reported by various sensor technologies, which may be incorporated in the sensors S. For example, the sensors S may include one or more of a blood oxygen sensor (e.g., optical micro light emitting diode [LED]), pressure and/or force sensors (e.g., piezoresistive, capacitive), a temperature sensor (e.g., piezoelectric transducer), or other micro to nano-scale sensors. In operation, the sensors S may be operable to measure and wirelessly communicate information related to pulse or respiration rate, blood oxygen level, blood pressure, site temperature, cavity or joint pressure, musculotendinous junction tension, intramuscular forces, fixation stability, relative position, stability or vibration of fixation devices (e.g., anchors, pins, nails, screws, buttons, sutures), and various related characteristic associated with a surgical site.

In various implementations, the sensors S may be configured to operate for extended periods without physical connection to a power source. In this way, the accessories 10 may be embedded or implanted in the anatomy of patients without requiring intermittent access to maintain operation. For example, the sensors S may be incorporated into one of more components, accessories, or portions of an implant system or surgical kit. Once implanted, the sensors S and corresponding control circuitry may receive or derive operating energy via inductive charging, wireless energy harvesting (e.g., ultrasound, electromagnetic), thermal or biomechanical energy harvesting, etc. to provide operating energy through an associated service life. Depending on the specific application, the proportions and monitoring features of the sensors S may be varied to suit size constraints and/or cost considerations associated with each application.

To effectuate the operability over an extended service life (e.g., multiple years), the control circuits of the sensors S may comprise wireless transmitters or transceivers, which may provide for wireless communication and charging via a local communication system associated with the monitoring system. In many cases, the sensors S may communicate wirelessly, such as by radio or Wi-Fi in a limited frequency range. The waveform may be communicated to activate and deactivate the sensor by causing a change in voltage. The sensors may incorporate pre-quantum technology to close the circuits and/or provide energy harvesting. An analog signal of a chip associated with the sensor may change in response to a change in the surrounding environment. The receiver may convert the signal from analog to digital.

In some implementations, the sensors S may emit a series of waves associated with the adjacent environment (e.g., anatomy). A controller of the monitoring system or compatible device may receive and monitor the waves to determine a change in the environment. For example, rather than communicating conventional messages or packets of data, the control circuit of each of the sensors S may output one or more signals (e.g., electromagnetic waves) that vary to indicate attributes of the environment local to the accessory 10 (e.g., the patient anatomy). In response to the signals from the control circuits of the sensors S, the controller of the monitoring system may monitor the signals from the sensors S for changes in amplitude, wavelength, wave propagation, electron entanglement, and/or other parameters to determine a change in attributes of the environment, such as a change in temperature and/or pressure of the anatomy. In this way, the sensors S may communicate various forms of information to report the condition of the anatomy local to the sensors S and/or the relative position of multiple sensors S, which may be indicative of the operation or efficacy of a surgical accessory 10 (e.g., an implant, suture, anchor, fastener, etc.)

As described in various implementations, the sensors S may provide for complex, active tracking or multiple moving bodies including anatomical portions of a patient relative to a surgical tool or instrument. For example, three or more sensors may be incorporated into an instrument to provide active tracking. Three sensors may establish a plane. The system may employ a transformation matrix of translation and rotation based on an array of sensors. More than three sensors may be utilized to reduce errors. For example, multiple portions (e.g., segments, accessories, fasteners, etc.) of an implant may be tracked and monitored independently to report complex kinematic movements of the musculoskeletal structure of a patient. In some implementations, multiple sensors may be used to establish a coordinate system between adjacent implants. Tracking an implant along one side of the joint may be utilized to determine ROM relative to another implant on an opposite side of the joint. The information may be utilized to make other determinations, such as Varus/Valgus. Additionally, the sensors S may track movements that may result from detrimental shifts in the position and/or orientation of portions of the implant. Such changes may represent failure conditions or separation from the anatomical structure of the patient.

The proposed system may track joints with sensors and may be utilized to determine the relation of the instrument relative to the joint. Sensor information may be utilized intraoperatively to assist the surgeon. The sensor information may be communicated to an augmented reality (AR) system. The surgeon may interact with the AR system interoperative to perform an orthopedic procedure. The AR system may be utilized to indicate a predetermined position, orientation, path, etc. of surgical devices utilized in the procedure, including instrumentation, implants and other devices. In order to report the respective locations and orientations, the sensors S may be positioned in connection with the corresponding anatomy to establish a visual representation of orientation of the joint. For example, sensors may be positioned to establish a three-dimensional representation of the scapula or humerus. A visual representation of the tissue may be generated in response to an insertion of a tool in the virtual joint. Both the anatomic structure of the patient and the position/orientation of the tool may be tracked in real time to update simulated graphical representations or maneuvering guidance in response to the movement of the patient and the tool.

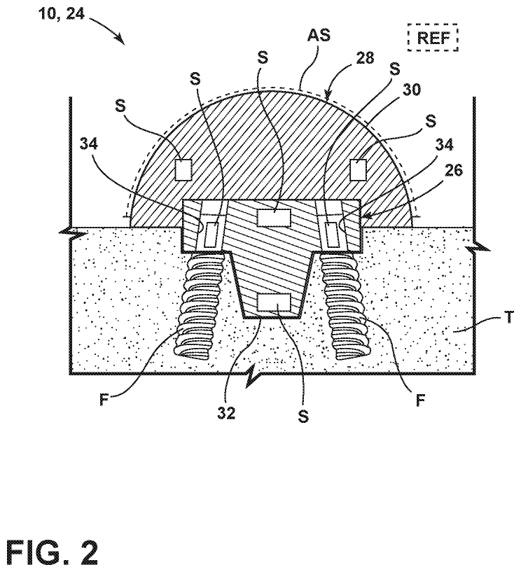

Referring generally to FIGS. 1 and 2, the sensors S may be utilized for verification in positioning a guide pin or an implant. For example, the surgeon may position one or more guide pins at selected landmarks of the patient anatomy. The landmarks may be used as a reference for positioning the guide and/or guide pins. Sensors may be utilized to provide a depth stop feature. Sensors may be incorporated into a drill bit and/or a respective guide to determine a depth of insertion (e.g., ream). FIG. 1 discloses a first device 20 and a second device 22 of a surgical system situated relative to tissue (e.g., bone) T of a patient. The first device 20 and second device 22 can incorporate any of the features disclosed herein. In some implementations, the first device 20 may be a guide. The second device 22 may be a cutting instrument (e.g., drill) or may be a positioning element (e.g., guide pin) that cooperates with the first device 20. In other implementations, the first device 20 may be a cutting instrument (e.g., drill), and the second device 22 may be a positioning element. The first device 20 and/or second device 22 may incorporate one or more sensors S. The sensors S may communicate with a common reference REF (e.g., receiver) of a monitoring system. The sensors S may be utilized to determine a relative position and/or orientation between the first device 20 and second device 22 relative to each other and/or the tissue T. The relative position may be utilized to determine a depth of insertion (e.g., ream) of the second device 22, which may serve as a depth stop feature to limit insertion of the first device 22 in the tissue T. The depth of insertion may be determined preoperatively and may be specified in a surgical plan. The sensors S may be incorporated at various positions of the first device 20 and/or second device 22.

In some implementations, one or more sensors S may be incorporated into a guide (e.g., the first device 20 or drill guide of FIG. 1) for implant positioning. A position of the guide may be determined with respect to a reference REF. The reference REF may correspond to an object with a fixed location relative to the tissue T of the patient. Accordingly, changes in the position and/orientation of the guide may be detected relative to the reference REF and the patient based on adjustments made by a surgeon. Similarly, the implant may incorporate one or more sensors (e.g., FIG. 2). A position of the implant may be determined with respect to the guide and/or may be tracked based directly on the position of the guide. The reference may be a guide pin received by, or spaced apart from, the guide. In implementations, the reference may be a receiver (e.g., FIGS. 1-2).

Sensors S may be distributed at various positions within the implant (e.g., FIG. 2) and may be utilized for relative positioning of components of the implant. For example, the implant may include a baseplate and a glenosphere. A pair of sensors S may be utilized to determine a lateralization and/or orientation of the glenosphere relative to the baseplate. In some implementations, sensors S may be incorporated into a set of anchors F (e.g., compression screws) for securing an implant. The sensors S may be utilized to determine a trajectory of the anchors F relative to the implant and/or anatomy. Sensors may be positioned in the anchors F and/or along a back of the implant to monitor temperature, pressure, blood flow, etc. The sensors may be utilized to determine a position and/or rotation of the implant.

The sensor information may be utilized to determine and monitor the health of the implant, the surrounding tissue, the position of the anchor(s) F and/or any associated devices. In various implementations, the sensor information may be utilized to measure changes in pH, blood flow, etc. of the adjacent environment. The changes may be compared to one or more predetermined thresholds to determine a likelihood of infection at the surgical site. A warning or other indicator may be generated to alert the surgeon in response to the change exceeding the predetermined threshold(s). The thresholds may be established relative to a baseline. The warning or other indicator may be generated to alert the surgeon in response to the change exceeding the predetermined threshold(s) for early evaluation and/or treatment of the patient.

FIG. 2 discloses an implant system 24 according to an implementation. The implant system 24 can incorporate any of the features disclosed herein. The system 24 can be utilized for various surgical procedures, such as an arthroplasty for restoring functionality of a joint. For example, the system 24 may be incorporated into a shoulder prosthesis and may be utilized to repair articular surfaces in an anatomical or reverse shoulder procedure. The system 24 may be utilized to restore functionality to other locations of the patient, such as knee and hip joints.

The system 24 may include a baseplate 26 and articulation member (e.g., glenosphere) 28. The articulation member 28 may be releasably secured to the baseplate 26 to establish an implant. The articulation member 28 may include an articulation surface 30. The articulation surface 30 may be dimensioned to cooperate with an opposed articular surface AS of an adjacent bone or implant (shown in dashed lines). The articulation surface 30 may have a generally convex or concave geometry complementary to a profile of the articular surface AS. In other implementations, the baseplate 26 may establish the articulation surface 30. The baseplate 26 may include at least one protrusion 32. The protrusion 32 may be an anchoring stem or keel. The protrusion 32 may be positioned in a recess in the tissue T to secure the baseplate 26 at the surgical site. The baseplate 26 may include one or more apertures 34. Each aperture 34 may be dimensioned to receive a respective fastener (e.g., anchor) F to fixedly attach or otherwise secure the baseplate 26 to bone or other tissue T. The fasteners F may be anchors or compression screws.

The implant system 24 may include one or more sensors S. The sensors S may be utilized according to any of the techniques disclosed herein. The sensors S may be utilized to determine a position and/or orientation of one or more components of the implant system 24 relative to each other and/or a common reference REF (e.g., receiver). In some implementations, sensors S may be incorporated into the baseplate 26, articulation member 28, and/or fasteners F. In some cases, a pair of sensors S may be utilized to determine a lateralization and/or orientation of the articulation member 28 relative to the baseplate 26. In various implementations, the sensors S may be utilized to determine a trajectory of the fasteners F relative to the baseplate 26 and/or patient anatomy. The sensors S may be utilized to determine a position and/or rotation of the baseplate 26, articulation member 28, and/or another component of the implant system 24 relative to each other and/or a common reference REF.

The sensor S information may be utilized to determine a change in component positioning, such as loosening of the component at the surgical site. In some implementations, the baseplate 26 may be secured to bone or other tissue T with a plurality of fasteners F. Each sensor S may be assigned a unique identifier (e.g., frequency). The sensors S may be positioned at a center of mass of the respective baseplate 26 and fasteners F. A change in distance between the centers of mass of the baseplate 26 and one of the fasteners F but not the remaining fasteners F may indicate loosening of the fastener F. A change in distance between the baseplate 26 and each of the fasteners F may indicate loosening of the baseplate 26. The change may be compared to one or more predetermined thresholds. A warning or other indicator may be generated to alert the surgeon in response to the change exceeding the predetermined threshold(s) for early evaluation and/or treatment of the patient. The thresholds may be established relative to a baseline.

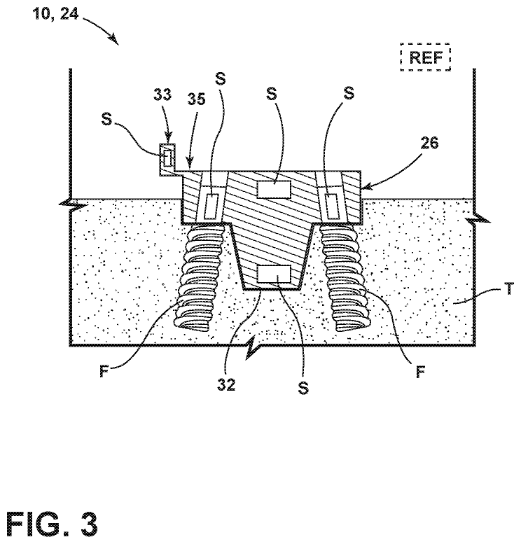

In some implementations, the sensors S may be incorporated into a removeable portion of the implant system 24. The removeable portion may be utilized to position one or more components of the implant system 24 relative to each other and/or the patient anatomy. In the implementation of FIG. 3, sensor(s) S may be incorporated into a carrier 33 coupled to the baseplate 26 or another portion of the implant system 24. The carrier 33 may be coupled to the baseplate 26 at a frangible connection 35. Once the surgeon positions and secures the baseplate 26, the frangible connection 35 may be severed to remove the sensor S and respective carrier 33. Information from sensor(s) S coupled to the carrier 33 may be communicated to an AR system for determining an interoperative position and/or orientation of the baseplate 26 relative to the anatomy and/or other components.

In implementations, an external bracelet or band may be worn by the patient and may be configured to interrogate one or more sensors, including any of the sensors S disclosed herein. The band may generate an alternating electric field. Sensors in the vicinity of the band may harvest energy from the electric field for powering the sensors. The band may be worn on the ankle for tracking movement of the tibia. The information may be utilized to model internal movement of a joint (e.g., a shoulder, an ankle, a knee).

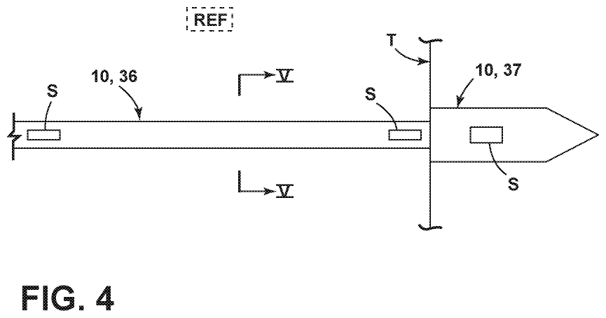

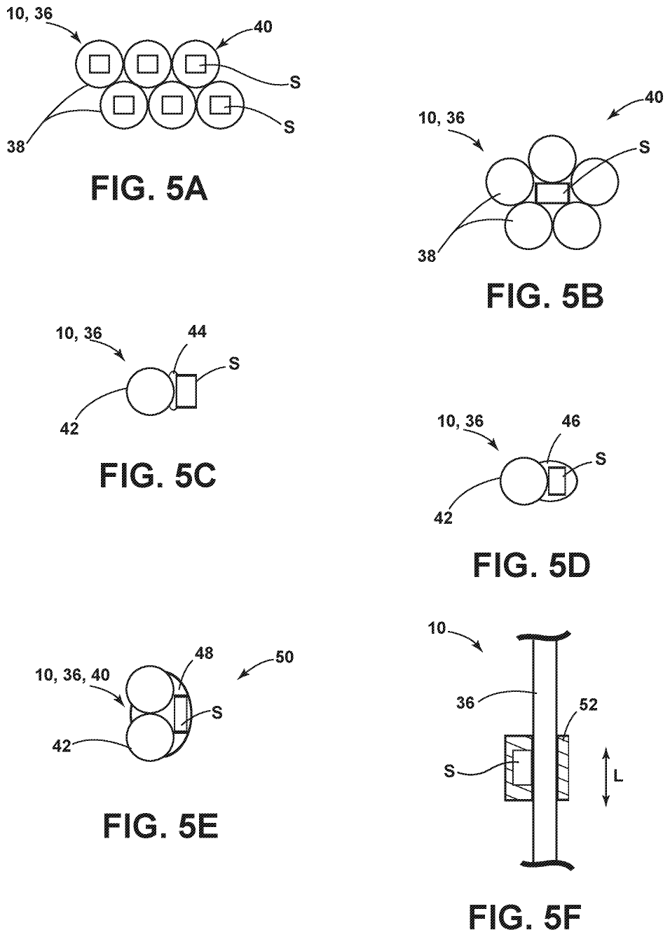

One or more sensors may be incorporated into suture to provide information (e.g., FIGS. 4-5). The suture may include a plurality of fibers. Sensors S may be incorporated at various positions of the suture. Sensors may be positioned along and/or within the suture construct. Sensors may be positioned in the fibers at different thickness and/or lengths of the suture. In an implementation, a set of sensors may be positioned at opposite lengths of the suture to determine a tension of the suture. In another implementation, a set of sensors may be axially aligned but may be incorporated into fibers at different thicknesses of the fiber stack. Sensors may be positioned between the fibers. The suture may be attached to one or more anchors. One or more sensors may be incorporated into the anchor to provide a reference for sensors incorporated into the suture. Sensors may be incorporated into a connector adapted to interconnect the suture and anchor. Sensor measurements may be utilized to determine tensile force and verify that the actual suture tension meets the preoperative plan and/or may be utilized to determine post-operative failure or degradation (e.g., comparing actual tension to a threshold value specified in the surgical plan).

Referring to FIG. 4, one or more sensors S may be incorporated into a length of suture 36 to provide information. The suture 36 may be secured to an anchor 37. The anchor 37 may be insertable in bone or other tissue T. In implementations, the anchor 37 may be a screw. One or more sensors S may be incorporated into the anchor 37. Referring to FIG. 5, the suture 36 may include one or more fibers 38. The fibers 38 may establish a fiber stack 40. One or more sensors S may be incorporated into, or otherwise positioned adjacent to, respective fibers 38. Sensors S may be positioned in a core of the respective fibers 38. The sensors S may be axially aligned along a common reference plane but may be incorporated into, or situated adjacent to, the fibers F at different thicknesses of the fiber stack 40.

Various techniques may be utilized to incorporate the sensors into the components. Each sensor may include a microchip. The microchip may incorporate a pre-quantum or other quantum architecture. In implementations, sensors may be incorporated into the components utilizing additive manufacturing. The sensors may be encapsulated in an insulated and/or waterproof construct. The sensors may be incorporated into a thickness or cavity of the component during fabrication.

Referring now to FIGS. 5A-5F, the sensors S may be secured to the suture 36 or similar thread-like structures and meshes in a variety of ways. As demonstrated in FIG. 5A, the fibers 38 of the suture may be woven or formed around a perimeter of the sensors S. In this configuration, one or more of a plurality of strands 42 forming the fiber stack 40 may include the sensors S embedded within the fibers 38 forming each of the strands 42. This configuration may allow the thread-like (e.g., round cross-section) or tape-like (e.g., rectangular cross-section) of the suture 36 or similar surgical accessories or connecting elements to incorporate the sensors S within an implanted assembly to provide the measurements and information reported to the monitoring system.

Additional configurations of the suture 36 or, more generally, connecting elements or features are demonstrated in FIGS. 5B-5F. As shown in FIG. 5B, the strands 42 of the suture 36 or connecting element may be woven together to enclose about the perimeter of the sensor S to form a pocket. Though not clearly shown in FIG. 5B, each of the strands may be braided, alternatingly interposed, and/or wound along a length of the suture 36 or connecting element. In this configuration, the pouch or pocket formed within the woven structure may enclose the sensor S allowing the suture 36 to be threaded or passed through tissue or connecting features to support a variety of applications. As demonstrated in various examples, one or more of the strands 42 may include the sensor(s) S affixed along a length. Additional strands 42 forming the suture 36 may be woven about the sensor(s) S to reinforce the suture 36 and/or provide a protective enclosure about the sensor(s) S.

As shown in FIGS. 5C-5E, the sensor(s) S may be connected to one or more strands 42 forming the suture 36 or connecting element of a surgical accessory 10. In the example of FIG. 5C, the sensor S is connected to a strand 42 via an adhesive 44. For example, the adhesive 44 may correspond to a bonding agent that connects a body of the sensor S to the strand 42. Similarly, as shown in FIG. 5D, the sensor S may be laminated within an adhesive or bonding layer 46, which may completely or at least partially encapsulate the sensor S and bond the sensor S to one or more strands 42 of the suture 36. In this configuration, the bonding layer 46 or lamination layer may bond the sensor S to the suture 36 and may enclose the sensor S providing a protective enclosure that may provide a smooth exterior surface allowing the suture 36 to be passed around or through tissue or connecting structures (e.g., eyelets, sheaths, etc.).

Referring now to FIG. 5E, the sensors S may be connected (e.g., adhered, laminated, over-molded, coated, chemically fused, thermally fused, etc.) to a plurality of the strands 42. In the example shown, a sealing layer 48 is utilized to connect the sensor S to the strands 42 forming the suture 36 or sensing element as well as arrange or interconnect the strands 42. In the example shown, the strands 42 in combination with the sensor S and the sealing layer 48 may form a cross-sectional perimeter that corresponds to a generally rectangular shape having a height greater than a lateral width. In some cases, the number of strands 42 and the arrangement may be adjusted, such that the suture 36 or connecting element defines different perimeter shapes 50. For example, the cross-section of the suture 36 may be elongated, rounded (e.g., similar to FIG. 5B), or otherwise shaped by grouping the strands 42 together in different arrangements with the sealing layer or bonding layer 48. Similar to the bonding layer 46 and adhesive 44, the sealing layer 48 may be implemented with various bonding adhesives suitable for biological applications. Variations in the perimeter shape 50 supported by the suture 36 may allow the resulting assembly with the sensor to have a reduced directional thickness, a directional flexion (e.g., bending, flexibility, maneuverability, etc.), and/or improve an exterior contour of the suture 36 to ensure that the perimeter 50 does not catch when being passed for various knots or connections.

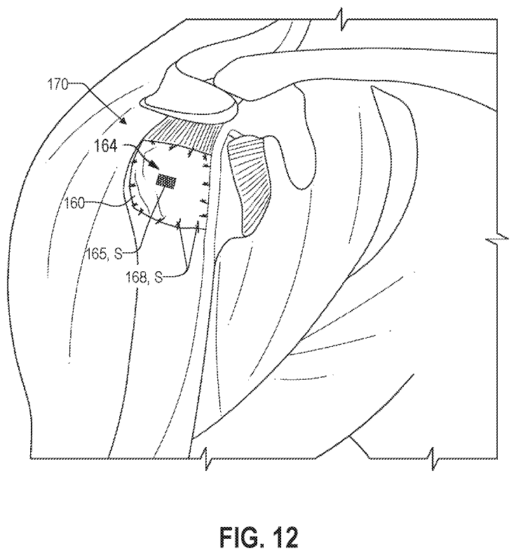

In the example shown in FIG. 5F, the sensor S may be connected to the strand 42 forming the suture 36 via an over-molded layer 52 that encloses the body of the sensor S as well as a portion of the strand 42. As shown, the over-molded layer 46 may enclose the entire perimeter of one or more strands 42 forming the suture 36 or connecting element, thereby encapsulating the body of the sensor S. As shown, the over-molded layer 52 may form a pocket or enclosure continuously extending about the suture 36 and the sensor S. Though demonstrated as having a rectangular cross-section, the over-molded layer 52 may correspond to an elongated structure that may taper along a length L of the suture 36. In this configuration, the sensor S may be secured within the over-molded layer 52 securing the sensor to the length of the suture 36. As later described in reference to FIG. 12, the sensor S may be implemented in various other implementations, such as a woven or mesh pocket 165. The pocket 165 may be connected to a graft or biological mesh and provide feedback as to various applied forces, a pressure, and/or a variety of characteristics.

Accordingly, the examples of FIGS. 5A-5F may provide for the sensors S to be adhered to or otherwise secured to an external surface of the suture 36. More generally, as demonstrated in the example of FIG. 12, the sensors S may be affixed to various fabricated components (e.g., a mesh, connecting feature (e.g., anchor, coupler, swiveling interconnection, joint), implants, and/or instruments to support various surgical procedures. Additionally, the sensors S may be connected or adjusted in position preoperatively and/or intraoperatively to ensure that the placement is aligned with one or more features of interest and/or anatomical structures of the patient. For example, in some implementations, one or more of the sensors S may be positioned on a length of tape, suture, or connecting element and the connecting element may be preoperatively or intraoperatively adhered at a position along an instrument or implant.

FIGS. 6A and 6B disclose various exemplary implementations of sensors for smart implants, instruments and other devices and associated methods. The features of FIGS. 6A and 6B may be used independently and/or together in accordance with any of the teachings disclosed herein. Referring first to FIG. 6A, the demonstrated implementation may provide for the tracking of the position and orientation of bones 80 (e.g., a femur 80a, a tibia 80b, and a fibula 80c) on opposing sides of a joint 82. As described in various examples, each of the bones 80 may comprise one or more of the sensors S rigidly attached or affixed via a surgical accessory 10 (e.g., an anchor, suture, implant component, etc.). By monitoring the positions and/or orientations of the sensors S relative to the reference REF, the monitoring system 90 may track the corresponding portions of the patient anatomy as well as one or more surgical instruments 84 or tools in a common coordinate system 86.

As shown in FIG. 6B, the system 90 may provide for a controller 92 comprising one or more monitoring circuits in communication with the one or more sensors S and transmission circuits (e.g., transceivers, emitters, etc.) configured to communicate the sensor data. In some implementations, the controller 92 may be in communication with or form a portion of one or more surgical consoles. The surgical consoles may be configured to communicate with and/or control a variety of corresponding surgical tools (e.g., shavers, drills, electrosurgical probes, pumps, etc.). In some cases, the monitoring system 90 may comprise a diagnostic computer (e.g., tablet, kiosk, base station, etc.) that may receive the sensor data from the accessories 10 for local diagnostics or remote monitoring. In general, the surgical accessories 10 or devices may be incorporated into instruments (e.g., 84) and/or implants comprising sensors and configured to detect or monitor various measurements, positions, orientations, and/or characteristics of the implant, accessory, or instrument reporting the status of the surgical site before, during, or after a surgical procedure. In general, the data reported by the sensors S may be monitored and/or recorded by the device controller 92, which may be provided in one or more of a wearable accessory, a surgical console, and/or a computerized device (e.g., tablet, computer, mobile device, etc.

As discussed herein, the controller 92 may be implemented with one or more processors 92a comprising one or more general purpose processors, microprocessors, microcontrollers, application specific integrated circuits (ASIC), field programmable gate arrays (FPGAs), computational processing units, graphic processing units, a group of processing components, or other suitable electronic processing components. The processor(s) 92a may be in communication with a memory 92b. In operation, the processor(s) 92a may execute program instructions, store, and/or access data in the memory 144 to perform the operations described herein. The controller may further comprise at least one communication circuit 92c that may be configured to communicate with the sensor(s) S or other related surgical/medical devices, databases, computers, etc. via one or more wired or wireless communication protocols. Some exemplary communication protocols may include (e.g., serial, Universal Serial Bus (USB), Universal Asynchronous Receiver/Transmitter (UART), etc.) and/or a wireless communication interface (e.g., a ZigBee, an Ultra-Wide Band (UWB), Radio Frequency Identification (RFID), infrared, Bluetooth®, Bluetooth® Low Energy (BLE), Near Field Communication (NFC), etc.) or similar communication standards or methods. The controller 92 may comprise or be in communication with one or more display devices 92d and/or user interfaces 92e to output indications, alerts, notifications, etc., as well as receive inputs from one or more users.

As shown, the controller 92 is also in communication with an external device, database, or server 94, which may correspond to a network, local or cloud-based server, device hub, central controller, or various devices that may be in communication with the controller 92 and, more generally, the system 90 via one or more wired (e.g., Ethernet) or wireless communication (e.g., Wi-Fi, 802.11 b/g/n, etc.) protocols. For example, the controller 92 may receive updates to the various modules and routines as well as access data for various surgical implements, accessories, procedures, sensors S, etc. from a remote server for improved compatibility, operation, diagnostics, and updates to the system 90.

Returning to the detailed example shown in FIG. 6B, the reference REF may correspond to a wearable device 88 affixed to a portion (e.g., an ankle) of the patient. In this configuration, the sensors S may communicate with the wearable device 88 as a common interface (e.g., receiver) of the monitoring system, which may establish a common coordinate system within which the orientations and positions of each of the bones 80, the joint 82, and instruments 84 may be tracked for various procedures. In this way, the disclosure may provide for the three-dimensional tracking of various features of the patient and/or surgical instrument by identifying the relative positions and orientations of one or more planes or local coordinate systems of the sensors S in the common coordinate system. Comparisons of information between two or more sensors, including any of the sensor arrangements disclosed herein, may be utilized in accordance with any of the features, measurements, and/or other teachings disclosed herein (e.g., position, orientation, temperature, pressure, blood flow, tension, etc.).

By detecting and tracking the positions of the sensors S, for example as depicted in FIG. 6, the monitoring system may detect and track the position and motion of the anatomy (e.g., bones 80, joints 82, etc.) of the patient. In operation, the monitoring system may process information from the sensors S situated relative to the patient to detect the range of motion (ROM) resulting from musculoskeletal movements of the patient. For example, the monitoring system may compare position and/or orientation data among two or more of the sensors S. In some cases, the sensors S may be distributed in an array relative to the joint 82 or another portion of the anatomy. The sensors S may be within the same implant or other device (see, e.g., FIGS. 2 and 4-5). The sensors S may be within different implants (see, e.g., FIG. 6). Comparisons between two or more sensors associated with a patient may be made with respect to the common coordinate system based on a common reference point (e.g., reference REF).

In general, the operations of the monitoring system and sensors S may be made in accordance with any of the features, measurements, and/or other teachings disclosed herein (e.g., position, orientation, temperature, pressure, blood flow, tension, etc.). For example, the sensor(s) S may be configured to monitor the area local to a surgical site for various conditions alone or in combination with the position and orientation. For example, the temperature and pressure local to the sensors S may be indicative of swelling or an infection that may result from complications in recovery. Such variations may be monitored by the systems and controllers (e.g., tablets, computers, base stations, wearable devices, etc.) described in the disclosure in relation to the position or orientation of the sensors S or independent of the positional data. For example, following patient discharge, a monitoring unit (e.g., a base station, tablet, handheld, wearable device, etc.) may interrogate the sensors S periodically to monitor changes in the local environment of each of the sensors (e.g., pressure, temperature, force, etc.). Alternatively, such conditions may be accessed via a care provider during a patient visit by activating (e.g., initializing or wirelessly communicating energy) the sensors S to diagnose or monitor conditions related to the surgical accessories 10 and corresponding patient anatomy local to the surgical site.

Based on the sensor data reported, the monitoring system may compare the environmental data to empirical data to determine whether a pressure or temperature is indicative of an infection or complication (e.g., failure, misalignment, release of fixation, etc.) of a surgical accessory or implant. Additionally, the monitoring system may document or store the data reported by the sensors S and associated with a patient over time to establish baseline values for one or more of the sensed characteristics (e.g., pressure, temperature, force, etc.) during an observation period. In response to the baseline values, the controller of the monitoring system may establish one or more predetermined ranges that may comprise thresholds (e.g., upper and lower thresholds) over which the sensor data reported is representative of expected values that may correspond to healthy tissue or conforming performance of a surgical accessory 10 or implant comprising one or more of the sensors S. In this way, the operation of the monitoring system may be personalized and tailored to each patient to ensure that physiological variations can be accounted for among a wide variety of applications and surgical procedures for a diverse patient population.

Following the observation period, the monitoring system may compare the sensor data to the baseline operation (e.g., the predetermined thresholds and/or ranges) for one or more of the sensed characteristics. In some cases, the thresholds and/or ranges may vary based on the relative position of the sensors S and the corresponding position of the patient (e.g., in response to an elevated appendage, sitting/standing positions, angle of joint, etc.). Additionally, the detection range may vary in response to one or more sensed characteristics associated with an activity of the individual that may be detected in response to an elevated heartrate, range of motion, relative elevations, rate, and extent of motion, etc. For example, the sensed characteristics reported by the sensors S (e.g., temperature, pressure, force, etc.) may vary in cases where the patient is exercising or participating in various activities. In order to account for such variations, the monitoring system may track the heartrate, respiration rate, movement, relative elevation, and other sensed characteristics to adjust the expected operating ranges or thresholds to which the sensed conditions (e.g., temperatures, pressures, forces, etc.) experienced by the sensors are compared to identify anomalous behavior of an implant (e.g., failure, misalignment, detachment, etc.) or complications related to a surgical procedure (e.g., swelling, infection, etc.).

In some cases, variations in pressure or force (e.g., tension, compression, etc.) detected by one of the sensors S may be indicative of a loss of fixation of an anchor 37 or a severed or slackened connection of a suture 36 or connecting element. The determination of such changes may be initially diagnosed in response to a detection of a change in the pressure, force, relative position among sensors, or other detected conditions identified by the monitoring system. For example, the operation of the sensors S may be interrogated and scanned by a controller (e.g., a tablet, a kiosk, wearable device, base station, etc.) that may receive the sensor data from the accessories 10 for local diagnostics or remote monitoring. In response to the detection of an anomalous condition (e.g., outside a predetermined or accepted range), the monitoring system may report the condition to a user or patient or remotely report the corresponding data to a remote health care provider. In this way, surgical treatment may be initiated by the monitoring system. Additionally, the monitoring system may be implemented as a diagnostic system to determine a source of pain or improper operation associated with an implant.

In general, the surgical accessories 10 or devices may be incorporated into instruments and/or implants to detect or monitor various measurements, positions, orientations, and/or characteristics of the implant or instrument reporting the status of the surgical site before, during, or after a surgical procedure. Referring to the examples shown in FIGS. 7 and 8, the data communicated from the sensors may be monitored over time or compared during a movement of a patient to identify changes in a relative position of the sensors S relative to one another. For example, the monitoring system may compare sensor information reported between or among two or more sensors positioned relative to a surgical accessory (e.g., a suture, anchor, pin, fixation device, etc.). As previously discussed in the examples of FIGS. 1-4, two or more sensors may be positioned within or otherwise relative to the same surgical accessory 10 (e.g., the suture 36, pin, anchor, or fixation device F, etc.) or anatomical structure.

For example, if two sensors S are affixed to different portions of a rigid structure (e.g., a bone), the data reported by the sensors may detect a change in a relative position of a first sensor that has become positionally decoupled from a second sensor. The corresponding sensor data may indicate that the position of the first sensor moved disproportionately to the position of the second sensor despite the intended rigid geometric relationship. For example, the controller may monitor the positions and/or orientations of the sensors to determine whether a directly proportional relationship is maintained in the changes in position reported by the sensors affixed, implanted, or otherwise attached to a rigid anatomical feature, implant, or construct. In some cases, the detected motion may correspond to a change in an absolute positional relationship. In response to such changes, a controller of the monitoring system may infer that a portion of the corresponding rigid structure has shifted, which may be reported as an indication of a misalignment or disconnection of the surgical accessory 10. In other cases, the change in the position of one or more of the sensors S may be detected in response to ongoing or periodic variations experienced by a first sensor connected to a rigid body but not a second sensor connected to the same rigid body. In such cases, the controller of the reporting system may similarly determine and output an indication that one of more of the surgical accessories 10 is no longer effectively attached to the rigid structure as determined by the disproportionate movement of one of the connected sensors S.

As shown in FIG. 7, two or more of the sensors S may be positioned within or otherwise relative to different structures or portions of the surgical accessories 10. Accordingly, the sensors S and operation of the monitoring system may provide for the detection of the relative motion of the position of one or more of the surgical accessories 10. In this way, the relative position and/or orientation of the surgical accessories 10 utilized to determine various conditions including the detachment, loosening, disconnection, or similar movement of one or more of the sensors S, which may cause the controller to identify that a rigid coupling among multiple sensors S and corresponding surgical accessories 10 is becoming detached or otherwise failing.

In the implementation of FIG. 7, sensors S are situated in or connected to sutures 106. The sutures 106 may be secured to one or more anchors 107. The anchors 107 may be secured to bone or other tissue T. The sutures 106 may be arranged to establish a suture structure (e.g., construct) SC. The suture structure SC may be utilized to secure soft tissue ST (FIG. 7). Comparisons of sensor information between two or more sensors S positioned within or otherwise relative to the same suture structure SC may be performed. Comparisons of sensor information of one suture 106 relative to another suture 106 may be performed. Comparisons of sensor information of one suture 106 relative to another suture 106 within the same suture structure SC may be performed. Comparisons of information between two or more sensors positioned relative to suture, including any of the sensor arrangements disclosed herein, may be utilized in accordance with any of the features, measurements and/or other teachings disclosed herein (e.g., position, orientation, temperature, pressure, blood flow, tension, etc.). The comparisons among the positions and/or orientations of the sutures 106 and, more generally, the surgical accessories may be detected by monitoring the data from the sensors S to determine variations in the relationship among the sensors S forming the suture structure SC or construct.

As demonstrated in FIG. 8, the data reported by the sensors S may similarly be applied to compare of sensor information reported among a plurality of anchors 107. In operation, the controller of the monitoring system may detect the relative movement of one of the anchors by detecting movement of one anchor relative to another anchor. As shown in the example of FIG. 8, an implant system 94 may correspond to a system for a glenoid implant comprising a baseplate 96 and articulation member (e.g., glenosphere) 98. The articulation member 98 may be releasably secured to the baseplate 96 to establish an implant. The articulation member 98 may include an articulation surface 100. By comparing the relative positions of the anchors 107, screws, and/or various fixation devices F as previously discussed, the performance and fixation of these surgical accessories 10 may be monitored. Additionally, comparisons of sensor information of one anchor relative to another anchor associated with the same suture structure may be performed as shown in FIG. 7. Further, comparisons of information between two or more sensors positioned relative to anchor(s), including any of the sensor arrangements disclosed herein, may be utilized in accordance with any of the features, measurements and/or other teachings disclosed herein (e.g., position, orientation, temperature, pressure, blood flow, tension, etc.).

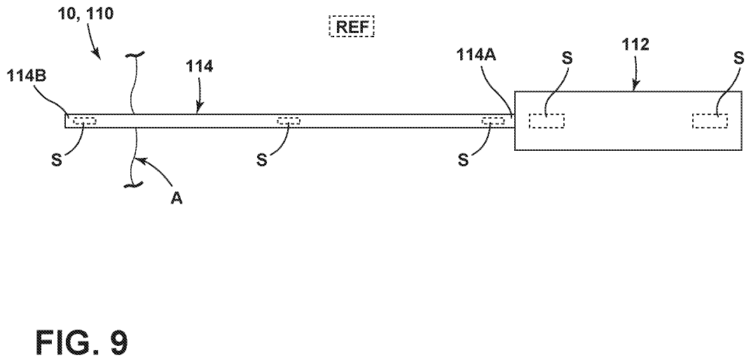

An instrument for an orthopedic or other surgical procedure may incorporate two or more sensors at different positions of the instrument (see, e.g., FIGS. 1, 6, and 9). Comparisons of sensor information from the sensors at the various positions of the instrument may be performed. In the implementation of FIG. 9, an instrument 110 is disclosed. The instrument 110 may incorporate any of the features of the devices disclosed herein. The instrument 110 may be positioned relative to a bone, joint and/or other anatomical feature A of the patient. The instrument 110 may include an instrument body 112 and a distally extending portion 114. In implementations, the distally extending portion 114 may be an elongated shaft. One or more sensors S may be incorporated into the instrument body 112 and/or distally extending portion 114. The distally extending portion 114 may extend axially between a proximal end 114A and a distal end 114B. Two or more sensors S may be positioned at different lengths of the distally extending portion 114 between the proximal and distal ends 114A, 114B. One or more sensors S may be positioned at, or otherwise adjacent to, the proximal end 114A. One or more sensors S may be positions at, or otherwise adjacent to, the distal end 114B of the distally extending portion 114, such as a terminal end of the distally extending portion 114. Comparisons of information between two or more sensors S positioned relative to instrument 110, including any of the sensor arrangements disclosed herein, may be utilized in accordance with any of the features, measurements, and/or other teachings disclosed herein (e.g., position, orientation, temperature, pressure, blood flow, tension, etc.).

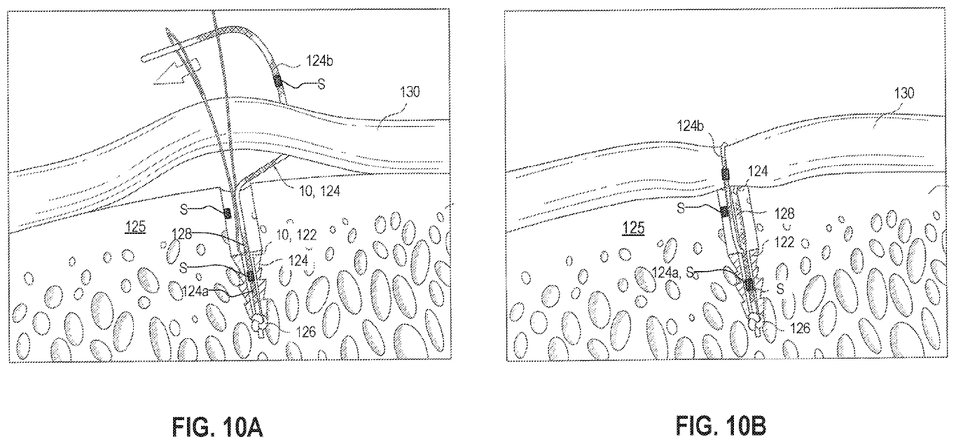

Referring now to FIGS. 10A and 10B, the operation of the sensors S and the surgical accessories 10 are described in reference to an exemplary surgical assembly. In the example shown, the sensors S are incorporated in surgical accessories 10 forming an assembly or kit for a knotless or self-cinching anchor 120. Though demonstrated as a knotless anchor, it shall be understood that various types of sutures, anchors, or other fixation assemblies may similarly be implemented, including knotted assemblies. As shown, the anchor 120 may comprise sensors S incorporated in or otherwise affixed to an anchor body 122, a first suture portion 124a, and a second suture portion 124b of a strand or suture 124. The sensor S of the anchor body 122 may be molded into or otherwise embedded in an opening within the material forming the anchor body 122 configured to engage a bone tunnel formed in a rigid structure or bone 125 of a patient. The sensors S incorporated in each of the first and second suture portion 124a, 124b may be incorporated in various connection configurations, some of which are demonstrated and discussed in detail in reference to FIGS. 5A-5F. In general, each of the sensors S may be configured to detect and report or communicate one or more of a temperature, pressure, blood flow, tension, etc. attributed to forces applied to the surgical accessories 10.

For example, the sensors S incorporated in connection with the suture 124 may be configured to measure and report the tension applied to the first suture portion 124a and the second suture portion 124b. The first suture portion 124a may be located on the suture 124 along a length disposed proximate to a knot 126 within an interior passage or cannula 128 formed through the anchor body 122. The sensor S in connection with the first suture portion 124a may report sensor data including a tension applied to the anchor 120. The second suture portion 124b may be located on the suture 124 along a length disposed along a closure loop 129 configured to enclose about tissue 130 outside the cannula 128. The sensor S in connection with the second suture portion 124b may report sensor data including a tension applied to the tissue 130 by the suture 124. The reporting of the tension or forces applied to the suture may be monitored by the system to ensure that the connection to the anchor 120 is maintained as well as detect occurrences where tension in excess of a threshold is applied to the anchor 120 or the tissue 130 as reported by each of the sensors S. As discussed in reference to the earlier examples, each of the sensors S may similarly be implemented to measure the position, orientation, temperature, pressure, blood flow, tension, etc.).

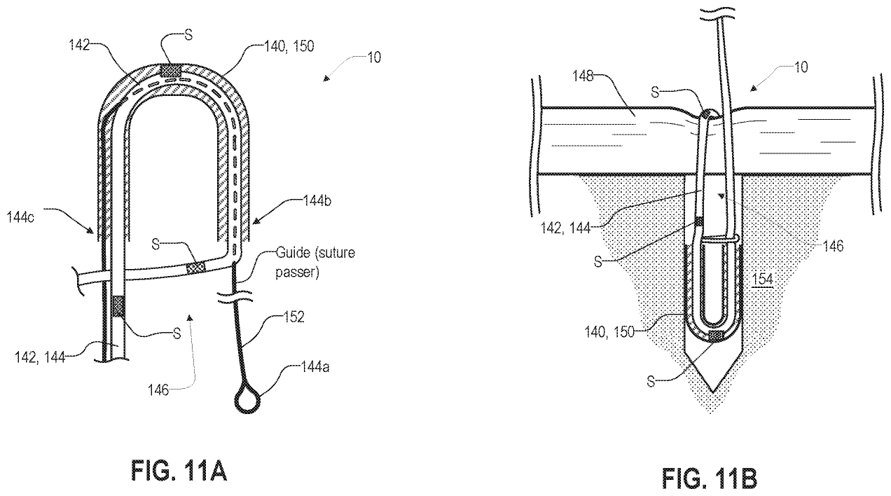

Referring now to FIGS. 11A and 11B, a similar example to that discussed in reference to FIGS. 10A and 10B is described in reference to a soft anchor 140 of a suture assembly. Though demonstrated as a knotless, soft anchor, it shall be understood that various types of sutures, anchors, or other fixation assemblies may similarly be implemented, including knotted assemblies. As shown, the soft anchor 140 may comprise a plurality of sensors S connected to a strand 142 forming a suture 144 utilized to form a flexible loop 146. A first sensors S may be incorporated in a section of the strand forming the loop 146, which may monitor the tension or pressure applied to enclosed tissue 148. A second sensor S may be incorporated in a sheath 150 that forms the body of the soft anchor 140. The sheath 150 may correspond to a mesh, which may include the sensor S within a pouch, pocket, or otherwise affixed to the sheath 150 that may provide for an anchored attachment. In operation, the strand 142 of the suture 144 may be passed through an eyelet 144a at a first end 144b of the cinching construct formed by the sheath 150. The strand 142 of the suture 144 may then be pulled through the sheath 150 by drawing a suture passer 152 back through a bone tunnel formed in an anatomical structure or bone 154 drawing the strand 142 through a second end 144c of the sheath 150. In this way, the sheath 150 may bind in the bone tunnel by cinching to form the soft anchor 140.

In general, each of the sensors S may provide for wireless communication with a controller identifying various characteristics of the surgical site and the surgical assembly including, but not limited to, a position, an orientation, a pressure, a force (e.g., tension, compression, directional forces), a temperature, a blood flow, a blood oxygen level, or other characteristics associated with a surgical site and efficacy of the surgical assembly. In the example shown, the sensor S may be incorporated in the sheath 150, which may be particularly beneficial in identifying a depth of the anchor 140 upon implantation and post-operative use as well as monitoring various characteristics (e.g., temperature, pressure, blood flow, tension, etc.) within a bone tunnel formed in a rigid structure or bone 152. In some implementations, a third sensor S may be positioned along the strand 142 proximate to the anchor 140 between the loop 146 and the anchor 140. The third sensor may be configured to monitor the tension or pressure applied between the tissue 148 and the soft anchor 140.

Referring now to FIG. 12, one or more sensors S may similarly be incorporated in a biological mesh 160 that may be formed by a plurality of fibers. As shown, the mesh 160 is implemented for a repair of a torn rotator cuff 164. The one or more sensors S may be connected via a woven or otherwise attached (e.g., adhesive, lamination, etc.) pocket 165. As shown, the pocket 165 may enclose about the sensor(s) S. Though a single assembly for the one or more sensor(s) S is depicted in the example shown, the mesh 160 may include a plurality of sensors S distributed about a surface area of the mesh 160. Additionally, one of more sutures 168 attaching the surgical mesh 160 to the tissue 170 may similarly include sensors S as discussed herein. In operation, the sensors S may report forces applied to the mesh 160, which may be monitored over time to determine the performance of the mesh 160 and the surrounding tissue 170. As discussed in various examples, changes to the forces detected by the sensors S in connection with the mesh 160 over time may be monitored by a monitoring device to identify variations that may be indicative of healing or later damage to the rotator cuff 164 and/or the surgical mesh 160. Accordingly, the sensor(s) S may be incorporated in the surgical device to monitor the effectiveness of the procedure and various characteristics that may be indicative of issues with fixation, stages of healing, and/or complications, such as infection.

As demonstrated in various examples throughout the disclosure, the sensors S may be implemented in a wide variety of surgical accessories 10 to provide for various sensory functions. In various applications, the sensors S may be applied in diverse configurations and combinations to monitor intraoperative routines and operations to provide guidance and/or feedback to a physician regarding the placement (e.g., position and orientation) of surgical accessory 10 (e.g., implant, fixation or fastening device, a surgical tool, etc.) to improve the placement and/or techniques associated with successfully effectuating a procedure. Intraoperative applications of the disclosed surgical accessories may include a verification of a placement of an implant or accessory relative to a patient anatomy as well as the monitoring of applied forces and conditions detected local to the surgical site. In various examples, the disclosed surgical accessories or devices may report position information from one or more implanted accessories as well as environmental information indicating the local conditions (e.g., temperature, pressure, blood flow, etc.) of the accessory and the localized environment. This information may be utilized to determine changes in the performance (e.g., fixation, rigidity, etc.) of the accessory as well as the local patient anatomy to which the accessory is attached. This information may assist health care providers in diagnosing patient conditions as well as improving the efficacy of procedures.

According to some aspects of the disclosure, an implant system for an orthopedic procedure comprises a first component including a first sensor. The first component forming a portion of a surgical assembly, and the first sensor is configured to detect at least one of a position, an orientation, a pressure, a force, a tension, compression, directional forces, a temperature, a blood flow, and a blood oxygen level. The sensor being in wireless communication with a controller configured to report sensor data detected by the first sensor

According to various aspects, the disclosure may implement one or more of the following features or configurations in various combinations:
a second component adjacent the first component including a second sensor;
the first sensor and the second sensor are in connection with at least one bone in spatially separated positions;
the first sensor is associated with a first bone of a joint, and the second sensor is associated with a second bone of the joint;
the first component is releasably secured to the second component;
the first component is a baseplate and the second component is a fastener configured to secure the baseplate to bone; and/or
at least one of the first component and the second component is an anchor, a fastener, a suture, a strand, a mesh, a fixation device, or portion or feature of an orthopedic assembly.

According to some further aspects of the disclosure, a surgical device for an orthopedic procedure comprises a main body and a sensor secured to the main body. The main body of the surgical device being at least one of an anchor, a fastener, a suture, a strand, a mesh, a fixation device, or a portion or feature of an orthopedic surgical assembly.

According to various aspects, the disclosure may implement one or more of the following features or configurations in various combinations:
the sensor includes a microchip, and the microchip incorporates a quantum architecture; and/or
the sensor is in communication with a controller that identifies a position of the sensor relative to a reference position.

According to some aspects of the disclosure, an apparatus for orthopedic surgery comprises at least one surgical accessory that connects to at least one anatomical structure. The at least one surgical accessory includes a first sensor and a second sensor connected to spatially separated portions of the at least one anatomical structure. A controller is in communication with the at least one surgical accessory and configured to detect a first position of the first sensor in a common coordinate system and a second position of the second sensor in the common coordinate system. The controller then identifies a spatial relationship between the first position and the second position and detects a change in the spatial relationship. Once detected, the controller outputs an indication reporting the change in the relationship as a diagnostic indication of a movement of a portion of the surgical accessory moved relative to the at least one anatomical structure.

According to various aspects, the disclosure may implement one or more of the following features or configurations in various combinations:
movement is identified as a change in a fixation state of the at least one surgical accessory to the anatomical structure;
change in the fixation state is a separation of the portion of the surgical accessory from the anatomical structure;
the spatial relationship defines a kinematic profile defining the relationship of the first position to the second position over a range of motion;
the spatial relationship is identified in response to a change in the kinematic profile resulting from one of the first position or the second position changing relative to the anatomical structure;
the first sensor and the second sensor are rigidly attached to the anatomical structure;
the anatomical structure is a continuous rigid structure;
at least one surgical accessory comprises a first surgical accessory comprising the first sensor and a second surgical accessory comprising the second sensor;
the first surgical accessory and the second surgical accessory are independently attached to the at least one anatomical structure;
the first surgical accessory and the second surgical accessory are interconnected by an orthopedic surgical assembly;
at least one fixation device comprises at least one of a pin, a button, a screw, an anchor, and a nail;
the surgical accessory comprises a suture or a flexible connecting element;
a third sensor in connection with a surgical instrument, wherein the controller is further configured to identify a third position in the common coordinate system;
each of the first sensor and the second sensor are configured to communicate with the controller via a wireless communication circuit;
the controller is further configured to distinguish first data communicated by the first sensor from second data communicated by the second sensor in response to a distinct signal characteristic or identification communicated to a receiver of the controller; and/or
at least one of the first sensor and the second sensor are configured to detect a temperature, a pressure, or a force applied to one or more of the spatially separated portions of the at least one surgical accessory.

According to another aspect of the disclosure, a method for detecting a condition of an orthopedic assembly comprises attaching the orthopedic assembly to a first portion of an anatomical structure with a first accessory including a first sensor and attaching the orthopedic assembly to a second portion, spatially separated from the first portion of the anatomical structure, with a second accessory including a second sensor. The method then identifies a first position of the first sensor in a common coordinate system and a second position of the second sensor in the common coordinate system. After determining a spatial relationship between the first position and the second position, the method is able to detect a change in the spatial relationship and a change in a fixation state of a surgical assembly relative to the anatomical structure in response to the change in the spatial relationship.

According to various aspects, the disclosure may implement one or more of the following features or configurations in various combinations:
the change in the fixation state indicates that a connection of the surgical assembly has at least partially decoupled from the anatomical structure;
the first sensor and the second sensor are rigidly attached to the anatomical structure;
the anatomical structure is a continuous rigid structure;
the first surgical accessory is at least one of a pin, a button, a screw, an anchor, and a nail;
the surgical accessory comprises a suture or a flexible connecting element;
the anatomical structure comprises at least one of a bone, a tendon, a ligament, a joint, a cartilage, and a muscle tissue; and/or
detecting at least one of a temperature, a pressure, or a force applied to one or more of the first portion and the second portion spatially separated portions of the at least one surgical accessory.

According to yet another aspect of the disclosure, a tissue repair apparatus comprises a fixation device comprising an anchor configured to engage a bone tunnel of a bone, the anchor comprising a body with an interior passage. A suture is in connection with the anchor and extends through the interior passage wherein the suture forms an enclosed loop that captures tissue and secures the tissue relative to the bone. At least one connection sensor is in connection with a portion of the suture. The at least one sensor is configured to detect a force applied to the suture and wirelessly communicate the force as sensor data.

According to various aspects, the disclosure may implement one or more of the following features or configuration in various combinations:
at least one connection sensor is positioned between the anchor and the enclosed loop and detects a tension applied between the anchor and the tissue;
at least one connection sensor is positioned on a portion of the enclosed loop and detects a tension applied about the tissue;
at least one connection sensor is woven into and enclosed by a plurality of strands forming the suture;
at least one connection sensor is enclosed within a pouch in connection with or formed along a portion of the suture;
at least one connection sensor is adhered to a portion of the suture;
at least one connection sensor is encapsulated within an over-molded structure formed about the connection sensor and a portion of the suture;
the body of the anchor is rigid or semi-rigid tubular body; and further comprising an anchor sensor disposed in a portion of the body;
the anchor sensor is configured to detect at least one of a position, orientation, a pressure, a force, or a temperature of the anchor and the surrounding tissue;
the anchor comprises a flexible mesh sheath forming the interior passage and further comprising an anchor sensor disposed in a portion of the body;
the connection sensor further configured to detect at least one of a position, orientation, a pressure, a force, or a temperature of the corresponding portion of the suture and the proximate tissue; and/or
a method for installing the tissue repair apparatus.

According to additional aspects of the disclosure, a tissue repair apparatus comprises a surgical accessory including at least one sensor configured for connection with an anatomical structure. The fixation device comprises at least one of an anchor, a fastener, a suture, a strand, a mesh, or a portion or feature of an orthopedic surgical assembly. At least one sensor is in connection with the surgical accessory and configured to detect at least one of a position, an orientation, a pressure, a tension force, a compression force, a directional force, a temperature, a blood flow, and a blood oxygen level. The sensor being in wireless communication with a controller configured to report sensor data detected by the first sensor

According to various aspects, the disclosure may implement one or more of the following features or configurations in various combinations:
the surgical accessory comprises a fixation device comprising an anchor configured to engage a bone tunnel of a bone forming the anatomical structure, the anchor comprising a body with an interior passage; and/or
a suture in connection with the anchor and extending through the interior passage wherein the suture forms an enclosed loop that captures tissue and secures the tissue relative to the bone.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

1.-10. (canceled)

11. An apparatus for orthopedic surgery comprising:
at least one surgical accessory that connects to at least one anatomical structure, the at least one surgical accessory comprising a first sensor and a second sensor connected to spatially separated portions of the at least one anatomical structure; and
a controller in communication with the at least one surgical accessory and configured to:
detect a first position of the first sensor in a common coordinate system;
detect a second position of the second sensor in the common coordinate system;
identify a spatial relationship between the first position and the second position;
detect a change in the spatial relationship; and
output an indication reporting the change in the spatial relationship indicating a movement of a portion of the surgical accessory relative to the at least one anatomical structure.

12. The apparatus according to claim 11, wherein the movement is identified as a change in a fixation state of the at least one surgical accessory to the anatomical structure.

13. The apparatus according to claim 12, wherein the change in the fixation state is a separation of the portion of the surgical accessory from the anatomical structure.

14. The apparatus according to claim 11, wherein the spatial relationship defines a kinematic profile defining the relationship of the first position to the second position over a range of motion and the change in the spatial relationship is identified in response to a change in the kinematic profile resulting from one of the first position or the second position changing relative to the anatomical structure.

15. (canceled)

16. The apparatus according to claim 11, any wherein the first sensor and the second sensor are rigidly attached to the anatomical structure.

17. The apparatus according to claim 11, wherein the anatomical structure is a continuous rigid structure.

18. The apparatus according to claim 11, wherein the at least one surgical accessory comprises a first surgical accessory comprising the first sensor and a second surgical accessory comprising the second sensor.

19. The apparatus according to claim 18, wherein the first surgical accessory and the second surgical accessory are independently attached to the at least one anatomical structure.

20. The apparatus according to claim 19, wherein the first surgical accessory and the second surgical accessory are interconnected by an orthopedic surgical assembly.

21. The apparatus according to claim 11, wherein the at least one fixation device comprises at least one of a pin, a button, a screw, an anchor, and a nail.

22. The apparatus according to claim 11, wherein the surgical accessory comprises a suture or a flexible connecting element.

23. The apparatus according to claim 11, further comprising:
a third sensor in connection with a surgical instrument, wherein the controller is further configured to identify a third position in the common coordinate system.

24. The apparatus according to claim 23, wherein each of the first sensor and the second sensor are configured to communicate with the controller via a wireless communication circuit and the controller is further configured to distinguish first data communicated by the first sensor from second data communicated by the second sensor in response to a distinct signal characteristic or identification communicated to a receiver of the controller.

25.-26. (canceled)

27. A method for detecting a condition of an orthopedic assembly, the method comprising:
attaching the orthopedic assembly to a first portion of an anatomical structure with a first accessory comprising a first sensor;
attaching the orthopedic assembly to a second portion, spatially separated from the first portion of the anatomical structure, with a second accessory comprising a second sensor;
identifying a first position of the first sensor in a common coordinate system;
identifying a second position of the second sensor in the common coordinate system;
determining a spatial relationship between the first position and the second position;
detecting a change in the spatial relationship; and
detecting a change in a fixation state of a surgical assembly relative to the anatomical structure in response to the change in the spatial relationship.

28. The method according to claim 27, wherein the change in the fixation state indicates that a connection of the surgical assembly has at least partially decoupled from the anatomical structure.

29. (canceled)

30. The method according to claim 27, wherein the anatomical structure is a continuous rigid structure.

31. The method according to claim 27, wherein the first surgical accessory is at least one of a pin, a button, a screw, an anchor, and a nail.

32. The method according to claim 27, wherein the surgical accessory comprises a suture or a flexible connecting element.

33. The method according to claim 27, wherein the anatomical structure comprises at least one of a bone, a tendon, a ligament, a joint, a cartilage, and a muscle tissue.

34.-50. (canceled)

51. An apparatus for orthopedic surgery comprising:
an orthopedic surgical assembly in connection with at least one anatomical structure comprising a first surgical accessory interconnected to a second surgical accessory, wherein:
the first surgical accessory comprises a first sensor and is in connection with a first portion of the at least one anatomical structure, and
the second surgical accessory comprises a second sensor and is in connection with a second portion of the at least one anatomical structure spatially separated from the first portion; and
a controller in communication with the at least one surgical accessory and configured to:
detect a first position of the first sensor in a common coordinate system;
detect a second position of the second sensor in the common coordinate system;
identify a spatial relationship between the first position and the second position;
detect a change in the spatial relationship;
compare the change in the spatial relationship to a known range defining a kinematic relationship between the first sensor and the second sensor;
in response to the change diverging from the known range, outputting a notification indicating the connection of the orthopedic surgical assembly has at least partially decoupled from the at least one anatomical structure.
